(19) United States
(12) Patent Application Publication
(10) Pub. No.: US 2019/0263226 A1
(43) Pub. Date: Aug. 29, 2019

(54) AIR PURIFICATION DEVICE

(71) Applicant: DR. SCHNEIDER KUNSTSTOFFWERKE GMBH, Kronach (DE)

(72) Inventors: Thomas GRUENBECK, Teuschnitz (DE); Gerhard ENDRES, Mainleus (DE); Jochen FIEDLER, Pressig (DE)

(21) Appl. No.: 16/334,260

(22) PCT Filed: Mar. 20, 2017
(86) PCT No.: PCT/EP2017/056523

§ 371 Date: Mar. 18, 2019

(30) Foreign Application Priority Data
Sep. 21, 2016 (DE) 10 2016 117 797.0
Oct. 28, 2016 (DE) 10 2016 120 656.3

(51) Int. Cl. B60H 3/06; A61L 9/20; B01D 46/00; B01D 53/88

(52) U.S. Cl. CPC B60H 3/0641; B60H 2003/0691; B01D 46/0036; B01D 53/885; A61L 2209/11; A61L 2209/14; A61L 2209/22; B01D 2253/102; B01D 2255/20707; B01D 2255/802; B01D 2257/708; B01D 2257/90; B01D 2258/06; B01D 2259/4566; A61L 9/205

(57) ABSTRACT

The present disclosure relates to a door having a door leaf with a plurality of door leaf sections which are connected in an articulated manner to one another by means of hinges, wherein a hinge has two hinge articulations of adjacent door leaf sections, at least one relatively long drive means which is connected to at least one door leaf section, and at least one guide means which is suitable for guiding the door leaf during its movement. In order to optimize the door in terms of its overall space, in particular in terms of width and depth and in so doing ensure a simple and cost-effective, but at the same time nevertheless reliable operation of the door, it is proposed to arrange the elongate drive means such that it is accommodated at least in certain portions in the door leaf section.

### FIELD OF THE DISCLOSURE

The present equipment serves for purification of air, particularly for purification of air in motor vehicle interior spaces.

### BACKGROUND OF THE DISCLOSURE

Various devices for air purification in motor vehicles are known from the prior art. These are mostly constructed and arranged in such a way that air is continuously extracted from the vehicle interior space, conducted across a filter and then fed back to the vehicle interior.

Thus, an air purifier is known from DE 10 2014 012 870 A1. This air purifier uses ultraviolet radiation for air purification. The air purifier comprises a housing with a housing inlet and a housing outlet, a fan arranged in the interior of the housing adjacent to the air inlet, an ultraviolet light-emitting diode unit and a filter unit. The filter unit is arranged in the interior of the housing above the fan along a flow path of the air. In addition, a flow arrangement arranged in the interior of the housing between the fan and the filter unit is also present. The flow control arrangement then controls the air flow along the flow path of the air between the outlet of the fan and the filter unit. In that case, photocatalytic ultraviolet-light-emitting diodes are employed for purifying the air.

A system for air purification with use of ozone and a ceramic, porous catalyser is disclosed in DE 20 2007 019 288 U1. The system comprises a housing, at least one inlet and outlet, at least one photon source, a ceramic core and a fluid flow generating device, wherein the photon source is arranged upstream of the ceramic core.

A device for purifying air is known from EP 0 707 989 A1. Purification is carried out in such a way that ambient air is inducted by way of a housing and a fan arranged therein, purified in a plurality of purification stages and then fed to the interior space of the vehicle. The fan or the drive of the fan is supplied with power by way of solar cells which are arranged in the vehicle and serve as energy sources.

It is disadvantageous with the aforesaid prior art that the purifying effect and, in particular, the elimination of noxious particles or odoriferous particles from the air to be purified are not adequately possible or possible over a longer period of time without maintenance intervention. This is due to the fact on the one hand that use is usually made of a mechanical filter which after a relatively short period of time has already taken up such a quantity of particles that it has to be exchanged in order to maintain the original purifying capability. If regular exchange is not carried out, such a filter contributes more to contamination of the air than purification thereof. It must be known to most users of air conditioning installations that a contaminated filter makes a substantial contribution to odour nuisance in a motor vehicle.

Moreover, it is disadvantageous that a filter change is connected with relatively high costs since usually such a change can be carried out only in a specialised workshop.

Moreover, it has to be taken into consideration that, in particular, new vehicles for a specific period of time deliver volatile organic compounds (VOC) and other substances hazardous to health. No systems are previously known from the prior art in order to eliminate this as promptly as possible and without burdening the occupants of a vehicle. Filter systems such as described in the introduction and also able to filter out such compounds are, in fact, present in the prior art, but the period of time over which these compounds remain in the vehicle is comparatively lengthy.

### SUMMARY OF THE DISCLOSURE

It is therefore an object of the present invention to eliminate the disadvantages of the prior art and to indicate an air purifying device which selectively reduces volatile organic substances and substances hazardous to health as well as other air contaminants in the air in the vehicle interior space and additionally also avoids and eliminates unpleasant odours.

This object is fulfilled by an air purifying device with the technical features indicated in claim 1. Advantageous developments are indicated in the subclaims, the further description and, in particular, the description on the basis of concrete embodiments.

The present invention relates to an air purifying unit with a housing having at least one inlet opening for the supply of an air flow and at least one outlet opening for discharging the air flow supplied by way of the inlet opening. At least one air purifying unit and at least one lighting unit are arranged in the housing. The at least one air purifying unit takes on substantially the form of the cross-section of the housing and is arranged in the housing to almost fill up the cross-sectional area. The at least one lighting unit is arranged in the housing to be opposite the at least one air purifying unit. The at least one air purifying unit is designed to be permeable to air and has on the side facing the lighting unit at least one photocatalytically active surface region or area region. Moreover, it has at least one region which is permeated or packed with activated carbon and which is at least partly surrounded by a non-woven material, wherein on the side of the at least one air purifying unit facing the lighting unit at least one photocatalytically active surface region is present in or at the non-woven material and the air flow in the housing at least in part flows around or through the at least one photocatalytically active surface region and the area region packed with activated carbon. The at least one photocatalytically active surface region of the at least one air purifying unit can be irradiated with light by the at least one lighting unit.

In an advantageous embodiment at least one area region packed or permeated with activated carbon is present on the side remote from the lighting unit. The air flow in the housing then flows at least in part through the at least one photocatalytically active surface region and the area region packed with activated carbon.

In a further advantageous embodiment of the invention the non-woven material is also packed or interspersed with activated carbon.

In an advantageous embodiment of the invention according to claim 2 it is provided that the at least one photocatalytically active surface region is at least partly coated with a metal oxide or a mixed oxide or is packed or permeated with a metal oxide or a mixed oxide.

By mixed oxide, also termed MOX or MOₓ for short, there is understood a substance which is composed of several oxides. There is a multiplicity of mixed oxides. By metal oxide there is understood a compound between a metal and an acid.

In an advantageous embodiment of the invention according to claim 3 it is provided that the metal oxide is an oxide of a transition metal. The chemical elements with the atomic numbers 21 to 30, 39 to 48, 57 to 80 and 89 to 112 are termed transition elements. Since these elements are all metals, these elements are also termed transition metals. This term “transition metals” is based on the position of these elements in the periodic system, since there the transition is shown by the successive increase of electrons in the d-atomic orbital along each period. Transition elements are defined by the IUPAC as elements which have an incomplete d-subshell or ions with an incomplete d-subshell.

In an advantageous embodiment of the invention according to claim 4 it is provided that the metal oxide or mixed oxide consists of CuO, Co₃O₄, CoOₓ, NiO, MnOₓ, MnO₂, MoO₃, ZnO, Fe₂O₃, WO₃, CeO₂, TiO₂, Al₂O₃, V₂O₃, ZrO₂, HfO₂, Dy₂O₃, Cr₂O₃, LiNbO₃ and/or Nb₂O₅.

In photochemical catalysers, as in the present case, individual oxides of transition metals can be used in the form of mixed oxides. These catalysers are active with oxides of transition metals and cause a complete and/or selective oxidation of volatile organic compounds.

The catalytic oxidation of volatile organic compounds by means of catalysers on the basis of noble metals is in part harmful for the structure thereof and the structural characteristics thereof. The effect of Pt (Pt=platinum) particle size on the catalytic oxidation of different hydrocarbons has shown that in general larger Pt particles are more active than smaller Pt particles. A lesser effect on the catalytic effectiveness of Pt catalysers is caused by factors such as the kind of carrier (aluminium oxide or silicon dioxide), the porosity and acid-base characteristics of the carrier. The addition of Co₃O₄, CeO₂, La³⁺/Bi³⁺ promoters to CeO₂-ZrO₂ leads to an increase in activity and thermal stability of catalysers with Pt and Pd (palladium) on the basis of aluminium oxide as well as to reduction of volatile organic compounds.

Noble metal catalysers such as Pt and Pd (palladium) exhibit good efficacy at low temperatures in complete oxidation of volatile organic compounds. The use of catalysers of that kind for catalysis of volatile organic compounds is limited with respect to the use in the interior space of vehicles due to high costs and technically-based risk of poisoning as a consequence of possible formation of chlorine and chlorine derivatives. By contrast, cerium oxide is very active due to its capability of oxygen enrichment. Oxidisation of volatile organic compounds to form CeO₂ is based on the redox mechanism. The modification of CeO₂ by other metal oxides, for example by partial replacement of Ce⁴⁺ ions by Zr⁴⁺ ions in the grid (mixed Ce—Zr oxides) can improve the oxygen capacity and thermal resistance of the catalyser as well as increase the catalytic activity at low resultant temperatures. An advantage of catalysers on a manganese basis is the high activity thereof for all oxidation reactions in conjunction with resultant low costs and toxicity. In addition, catalysers with the perovskite structure can be used.

Very good catalytic properties are also achieved by perovskite structures with the general formula thereof of ABO₃. In this formula, A is the element of rare earths and B is a transition metal.

In an embodiment of the invention which can be realised particularly satisfactorily the at least one photocatalytically active surface region is at least in part coated with titanium dioxide (TiO₂) or packed or permeated with titanium dioxide ions (TiO₂ ions). However, any other of the aforesaid compounds which are photocatalytically active can also be used.

In an advantageous embodiment of the invention according to claim 5 it is provided that the at least one air purifying unit is arranged at or fixable to the inner walls of the housing. Through the arrangement or fixing of the at least one air purifying unit at or to one of the inner walls of the housing it is possible to achieve good securing and at the same time the air flow in the housing can be guided so that it is optimally matched to the at least one air purifying unit. In the case of arrangement in the centre of the housing the at least one air purifying unit can be directly surrounded by the air flow flowing into the housing and a good purifying performance is achieved.

In an advantageous embodiment of the invention according to claim 6, it is provided that the at least one lighting unit is arranged in the housing of the at least one air purifying unit to be opposite the at least one photocatalytically active surface region of the at least one air purifying unit on one of the inner walls of the housing or almost in the middle of the housing. The inner walls of the housing are coated in the region between the at least one air purifying unit and the at least one lighting unit with a light-reflective material, particularly an ultraviolet-light-reflective material.

The lighting unit is absolutely necessary, since only through light, preferably ultraviolet light, incident on the at least one air purifying unit or the photocatalytically active surface regions thereof is the photocatalytic purifying process set in motion. Consequently, good illumination and thus feed of ultraviolet light are possible through the arrangement of the at least one lighting unit opposite the at least one air purifying unit.

In an advantageous embodiment of the invention it is provided that the at least one lighting unit consists of at least one light-emitting diode which emits ultraviolet light. Activation by visible light can also be carried out depending on the respective doping of the photocatalyser.

In an advantageous embodiment of the invention according to claim 7 it is provided that lenses which focus the light emitted by the at least one lighting unit onto the at least one air purifying unit are arranged in front of the at least one lighting unit, or light reflectors or light-reflective regions which reflect the light emitted by the at least one lighting unit in the direction of the at least one air purifying unit are arranged behind and/or adjacent to the at least one lighting unit. Through the arrangement of the lenses, a particularly good focusing of the emitted light onto the at least one air purifying unit is achieved. Through the arrangement of light reflectors or light-reflective regions it is achieved that even light which is not directed directly onto the at least one air purifying unit is nevertheless conducted at least partly to the air purifying unit.

In an advantageous embodiment of the invention according to claim 8 it is provided that the inner walls of the housing are painted white or coated with a light-reflective material. It is thereby achieved that the light emitted by the at least one lighting unit is not absorbed by the housing, but reflected and thus additionally conducted onto or in the direction of the at least one air purifying unit.

In an advantageous embodiment of the invention according to claim 9 it is provided that air guide elements deflecting the air flow diffusely in the housing are arranged in the housing so that the air flow impinges almost uniformly on the entire surface of the non-woven material of the at least one air purifying unit and flows therethrough or the air flow is directed for the major part onto the at least one photocatalytically active surface region and flows around or through this. In addition, desired turbulence of the air is thereby created. A particularly high purifying effect is thus achieved.

In an advantageous embodiment of the invention according to claim 10 it is provided that the at least one air purifying unit consists of a frame of plastics material or metal which is enclosed by the non-woven material at least at one side and a receiving region for granulate materials is present behind the non-woven material, wherein this receiving region is coated with activated carbon or a mixture of activated carbon and/or with titanium dioxide (TiO₂) and/or filled with granulate packed with titanium dioxide ions (TiO₂).

Thus, through the photocatalytic process which is triggered when light is incident on titanium dioxide, the activated carbon region can also be purified so that the filter achieves its full purifying capacity over a longer period of time. Moreover, contaminants adhering to the non-woven material can thereby be removed or eliminated.

In an advantageous embodiment of the invention it is provided that at least in part threads or fibres doped with titanium dioxide ions (TiO₂ ions) are worked or introduced into the non-woven material, which forms the at least one photocatalytically active surface region, or that regions of the non-woven material are doped with titanium dioxide ions (TiO₂ ions). Through the photocatalytic process activated when light is incident on titanium dioxide not only the air flowing past is purified, but at the same time the activated carbon region and contaminants adhering to the non-woven material are removed so that the filter achieves its full purifying capacity over a longer period of time.

In an advantageous embodiment of the invention according to claim 12 it is provided that the surface of the at least one air purifying unit with the non-woven material is formed to be wave-shaped or has conical, folded, cylindrical, frusto-conical, frusto-pyramidal, spherical or hemispherical geometric shapings at least at the side having the at least one photocatalytically active surface region. Through this configuration the surface, which is active for air purification, of the at least one air purifying unit can be increased. The specific shaping of the surface leads to only a small increase in air resistance opposing the air flow in the housing.

In an advantageous embodiment of the invention according to claim 13 it is provided that the at least one lighting unit is controllable in the light intensity thereof, wherein an air quality measuring sensor arranged in the air inlet channel or at the air outlet channel determines the air quality and a control unit controls the light intensity of the at least one lighting unit on the basis of the air quality determined by the air quality measuring sensor. Thus, on the one hand the electrical power absorption can be controlled appropriately to requirements and at the same time it is possible to avoid the at least one unit being operated continuously, even when not required, at maximum electrical load. This makes possible at the same time an extension of the service life of the at least one lighting unit.

In an advantageous embodiment of the invention according to claim 14 it is provided that the at least one lighting unit is so controllable in the light intensity thereof by the control unit that the control unit controls the light intensity of the at least one lighting unit in dependence on the rotational speed of a fan arranged in front of the air inlet channel or the air outlet channel. Thus, on the one hand the electrical power absorption can be controlled appropriately to requirements and at the same time it is possible to avoid the at least one lighting unit being operated continuously, even when not required, at maximum electrical load. The at least one lighting unit is controlled in its intensity in dependence on the amount of air flowing through the housing, thus a control appropriate to requirements. This additionally makes possible extension of the service life of the at least one lighting unit, since this is not always operated at maximum load.

In an advantageous embodiment of the invention according to claim 15 it is provided that the air purifying unit is arranged in or at the air intake of the air circulation flap of a motor vehicle. In this case an individual fan for the air purifying unit is not required, since the air flow of the air circulation operation can be exploited and conjunctively utilised.

In an advantageous embodiment of the invention according to claim 16 it is provided that a volatile organic compound sensor for control of the air purifying device appropriate to requirements is arranged in this or that a CO₂ sensor already present in the motor vehicle is incorporated for control of the air purifying device appropriate to requirements.

The air purifying device according to the invention is described in the following on the basis of actual embodiments with reference to figures. The following description on the basis of actual embodiments does not represent limitation of the invention to any one of these actual embodiments.

### BRIEF DESCRIPTION OF THE DRAWINGS

In the figures:

FIG. 1 shows a schematic construction of an air purifying device according to the invention;

FIG. 2 shows a perspective view of air purifying device; and

FIG. 3 shows a view of an air purifying device according to the invention with the further relevant components.

### DETAILED DESCRIPTION

The same parts and/or components are provided with the same reference numerals in the figures. These parts and/or components substantially correspond with one another insofar as nothing to the contrary is indicated.

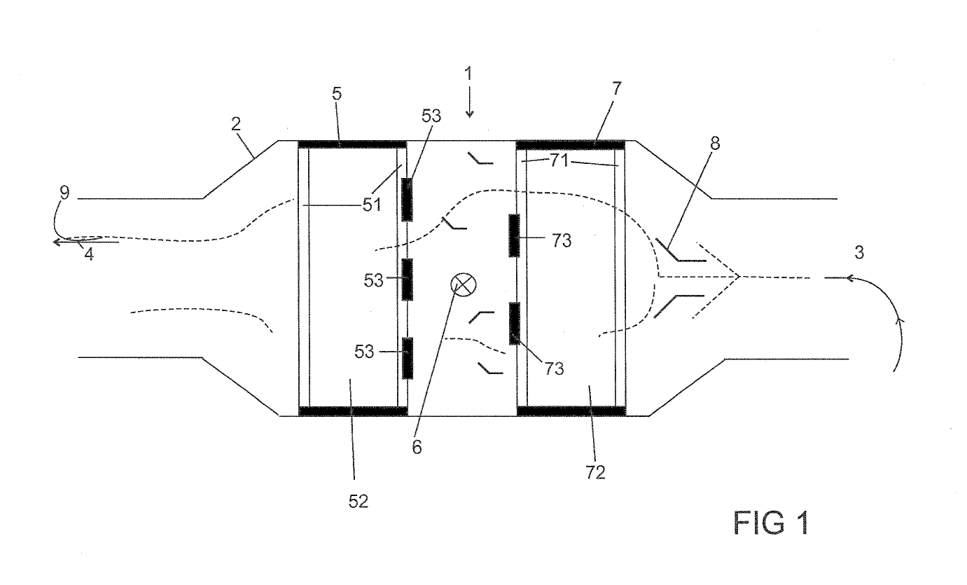

A section through a schematic air purifying device 1 is illustrated in FIG. 1. The air purifying device 1 has a housing 2. The form of the housing 2 can have a round, oval, hexagonal, polygonal or, preferably, rectangular cross-section.

Configuration of the cross-section of the housing 2 in a rectangular form has proved particularly advantageous. The housing 2 is block-shaped.

The housing 2 preferably consists of plastics material; in a special embodiment, the selected plastics material is ABS.

The housing 2 has, at two mutually opposite ends, an inlet opening in the form of an air inlet channel 3 and an outlet opening in the form of an air outlet channel 4 for air supplied to the housing 2 by way of the air inlet channel 3.

Air to be purified is supplied by way of the air inlet channel 3 to the air purifying device 1 into the housing 2 thereof; the air purified in the air purifying device 1 is conducted out of the housing 2 again by way of the air outlet channel 4.

An air flow 9 forms in the housing 2. The air flow 9 runs through the housing 2 from the air inlet channel 3 to the air outlet channel 4. The air flow is directed in the housing 2.

Two air purifying units 5, 7 are arranged in the housing 2. However, further air purifying units 5 can also be arranged in the housing 2 or, however, only a single air purifying unit.

The air purifying units 5, 7 are arranged at one of the inner walls of the housing 2 and are mechanically positively or frictionally connectible with the inner wall. For that purpose, clips or mounts which fix the air purifying units 5, 7 to the inner wall of the housing 2 are provided.

A lighting unit 6 is arranged between the two air purifying units 5, 7. However, a plurality of lighting units 6 can also be arranged in the housing 2.

The lighting unit 6 is so arranged almost centrally in the housing 2 or at the inner wall of the housing 2 that the light emitted by the lighting unit 6 is delivered so as to be directed almost entirely onto the air purifying unit 5 and the air purifying unit 7.

In an advantageous embodiment of the invention, lenses focusing the light of the lighting unit 6 onto the air purifying units 5, 7 are arranged in front of the lighting unit 6.

In a further embodiment of the invention prisms or mirrors which prevent the delivered light of the lighting unit 6 from not impinging on the air purifying units 5, 7 are arranged at the lighting unit 6.

In a further advantageous embodiment of the invention the interior of the housing 2 is painted white or provided with a light-reflective surface coating.

The air purifying units 5, 7 each have at least one photocatalytically active region 53, 73. In the embodiment according to FIG. 1 three regions 53 of that kind are present at the air purifying units 5 and two regions 73 of that kind are present at the air purifying units 7.

The photocatalytically active regions 53, 73 in the concrete embodiment according to FIG. 1 consist of titanium dioxide (TiO₂) or are doped or packed or permeated with titanium dioxide ions (TiO₂ ions). However, photocatalytically active materials other than TiO₂ can also be used, particularly the already mentioned materials, especially a metal oxide or mixed oxide of CuO, Co₃O₄, CoOₓ, NiO, MnOₓ, MnO₂, MoO₃, ZnO, Fe₂O₃, WO₃, CeO₂, TiO₂, Al₂O₃, V₂O₃, ZrO₂, HfO₂, Dy₂O₃, Cr₂O₃, LiNbO₃ and/or Nb₂O₅. However, other photocatalytically active substances and/or compounds can also be used. The afore-mentioned list is not definitive.

The air purifying units 5, 7 consist of a frame and a non-woven material 51, 53, which encloses the frame at least partly, and adjoins an interior space in which activated carbon is arranged or which is packed with activated carbon. This region permeated or packed or able to be packed with activated carbon is provided with the reference numeral 52, 72.

The lighting unit 6 is now tailored to the photocatalytically active regions 53, 73. The lighting unit 6 delivers ultraviolet light or visible light with a presettable or preset wavelength. The photons with the corresponding wavelength of the ultraviolet light or visible light of the lighting unit 6 when impinging on the respective photocatalytically active region 53, 73 trigger a photochemical reaction in the titanium oxide, which has the consequence that odiferous particles and/or noxious particles in the air are converted or destroyed. The odiferous particles and/or noxious particles impinging on the photocatalytically active regions 53, 73 are destroyed or converted by the photochemical process and the supplied air is thus purified.

The lighting unit 6 is preferably at least one light-emitting diode, preferably a UV-light-emitting diode. UV has the meaning ultraviolet.

In an advantageous embodiment of the invention a plurality of ultraviolet-light-emitting diodes is present and forms the lighting unit 6. The light-emitting diodes are arranged with respect to one another in a row to be equidistant. Several rows of ultraviolet-light-emitting diodes are then arranged in parallel adjacent to one another and at the same spacing from one another.

The lighting unit 6 is controlled by way of a control unit, which is not illustrated in FIG. 1.

Air guide elements 8 are arranged in the housing 2. These serve the purpose of guiding the air flow 9 in the housing 2 onto the air purifying units 5, 7 and the photocatalytically active regions 53, 73 so that a largest possible part of the air flow 9 with the noxious particles and/or harmful substance particles passes to the photocatalytically active regions 53, 73 so as to photocatalytically react thereat. In order to achieve a best possible flow of air around the photocatalytically active regions 53, 73 of the air purifying units 5, 7 and at the same time to not impair the light delivery of the lighting unit 6 to the air purifying units 5 the air guide elements 8 are arranged in front of and behind the lighting unit 6. In an advantageous embodiment the air guide elements 8 are provided with an ultraviolet-light-reflective coating.

The air flowing through the housing 2 is accordingly purified by the air purifying units 5, 7 and the photocatalytically active regions 53, 73 thereof when ultraviolet light radiated by the lighting unit 6 impinges thereon.

Each air purifying unit 5, 7 has a frame which is enclosed by a non-woven material 51, 71 and thus bounds an interior space 52, 72. Activated carbon or a granulate of activated carbon and/or a mixture of activated carbon and a granulate consisting of granulate doped with or permeated by titanium dioxide is filled into the interior space 52, 72. Iron particles or plastics material particles can be used as a carrier of the granulate for the titanium oxide ions. On the side of the air purifying unit 5, 7 associated with the lighting unit 6 the non-woven material 51, 71 arranged thereat is furnished with a photocatalytically active surface region 53, 73. The photocatalytic and photocatalytically active surface region 53, 73 is packed or permeated with titanium dioxide ions. The photocatalytic process is set in motion by the incidence of ultraviolet light or light on these regions 53, 73. Noxious substances conducted past in company with the air flow 9 are correspondingly reduced, destroyed or dissolved.

In order to form the photocatalytically active surface regions 53, 73 the non-woven material 51, 71 is, at these locations, at least partly doped with or permeated by titanium oxide ions or, however, threads or fibres doped with or permeated by titanium oxide ions are introduced or woven into the non-woven material 51, 71 or connected therewith. Alternatively, the doping with titanium oxide ions or the formation of these photocatalytically active regions 53, 73 can also be carried out in that the non-woven material is immersed in a liquid, in which titanium oxide ions are present or dissolved, or is sprayed with or wetted by that liquid.

The non-woven material 51, 71 is formed in such a way that at the same time it serves as a filter. In that case, the structure of a HERA filter can, in particular, serve as a template for the non-woven material 51, 71 so that a cleaning effect of coarse particles inducted together with the air flow takes place as well. In that regard it is advantageous that the non-woven material 51, 71 is arranged at both sides at the air purifying unit 5, 7 so that the entering air flow 9 is already subjected to preliminary cleaning by the non-woven material 71 and coarse particles are filtered out of the air flow.

Moreover, it is advantageous to form the surface of the non-woven material 51, 71 and thus the photocatalytically active regions 53, 73 to be of large area. It has therefore proved to be advantageous to enlarge the surface by folding or deforming, for example by forming a wave shape. In addition, it is also possible to form this surface with conical, folded, cylindrical, frusto-conical, frusto-pyramidal, spherical or hemispherical geometric shaped portions.

A further advantage of the invention is to be seen in the fact that through the photocatalytically active regions 53, 73 not only the air, but also the adjoining regions are purified therewith. Thus, a cleaning action on the entire non-woven material 51, 71 and also on the activated carbon arranged therebehind can take place at the same time.

It is particularly advantageous, as already mentioned, if the titanium oxide or titanium oxide ions are also directly incorporated in the form of granulate or in another form in the activated carbon region 52, 72. As a result, when the titanium oxide ions are activated by way of the photocatalytic process, the activated carbon is itself appropriately cleaned as well.

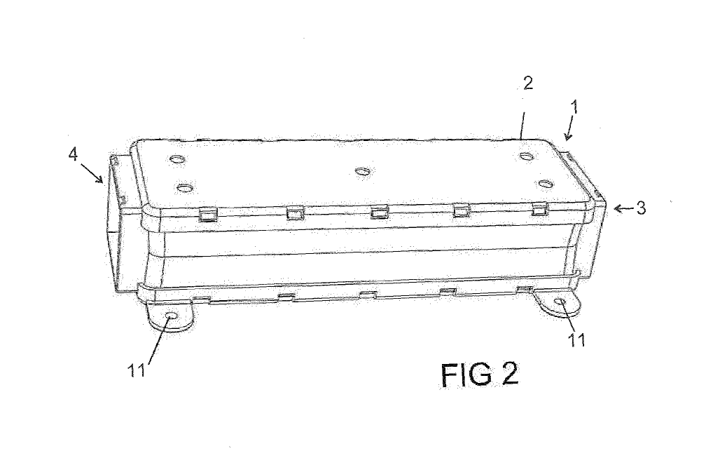

The housing 2 of the air purifying device 1 is illustrated in perspective view in FIG. 2. The housing 2 consists of a plurality of individual parts which can be assembled to form the housing 2. The housing 2 is of block-shaped form and has towards one side a narrowed portion which goes over into the air inlet channel 3. Provided on the side of the housing 2 opposite the air inlet channel 3 is a further corresponding narrowed portion which forms the air outlet channel 4. The air to be purified is removed from the motor vehicle interior space, in particular sucked therefrom, and supplied by way of the air inlet channel 3 to the housing 2 and thus the air purifying device 1. Purification of the air then takes place in the housing 2 in a manner analogous to that described for FIG. 1 and the purified air is then discharged by way of the air outlet channel 4 and fed back to the vehicle interior space.

In addition, mounts 11 which are fixedly connected with the housing 2 are present at the housing. These mounts 11 serve the purpose of being able to secure the air purifying device 1 in a motor vehicle at an intended location. For that purpose, a clamping connection or a screw connection is provided.

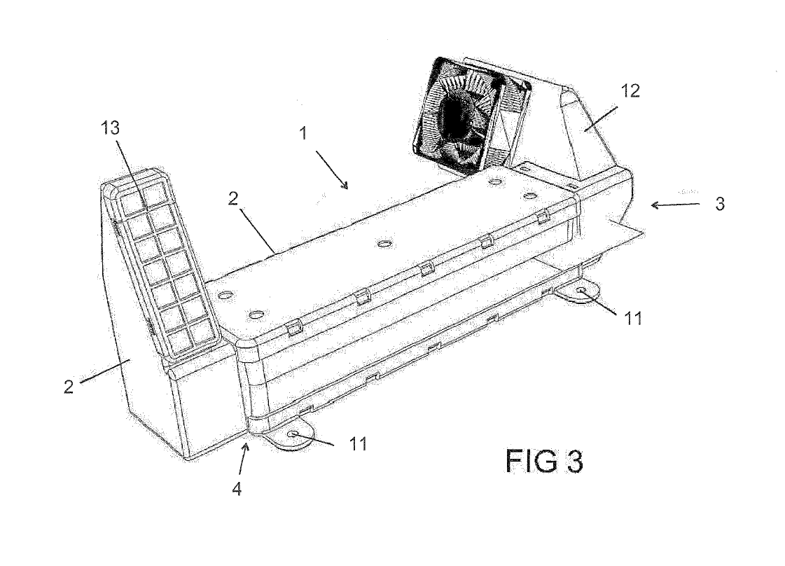

The air purifying device 1 with the housing 2 is illustrated in FIG. 3. Arranged at the air inlet channel 3 is an air feed channel 12, at the inlet of which a fan is arranged which sucks the air out of the vehicle interior space and blows it through the air feed channel 12 and via the air inlet channel 3 into the housing 2 of the air purifying device 1. The air feed channel 12 is plugged onto the narrowed portion of the air inlet channel 3 and is mechanically positively connected therewith.

In a special embodiment of the invention the air feed channel 12 is connectible with the air inlet channel 3 or the housing 2 by means of a snap connection. Arranged on the side of the air outlet opening 4 is an air guide channel 13 which receives the outflowing air from the air outlet opening 4, channels the air and correspondingly feeds it back to the interior space of a motor vehicle from which the air was sucked by means of the fan 11.

In a further advantageous embodiment of the invention it is provided that at least one further air filter unit such as, for example, a HEPA filter or another form of air filter is arranged in the air guide channel 12.

The housing 2 has a housing base 4 and a housing cover, which together form the housing 2. The mounts 11 are arranged at the housing base. Both the housing base and the housing cover have, laterally at the outer side, a plurality of indentations into which corresponding lugs at the housing side parts snap and thus connect the housing base and the housing cover by way of the housing side parts.

The air purifying units 5, 7 are arranged on the inner side of the housing cover and the inner side of the housing base and fixed thereat. Each air purifying unit 5, 7 is mechanically positively and/or frictionally connected with the housing base or the housing cover. This connection is effected by way of, for example, clipping, glueing or screw-connecting.

The lighting unit is one or more light-emitting diodes which emit ultraviolet light and are preferably tailored to the titanium dioxide and to the photocatalytic action thereof thus able to be produced and which have a wavelength in the range between 250 and 400 nanometres, preferably in the region around 367 nanometres. In the case of use of doped TiO₂, use can be made of visible light in the range of 400 to 500 nanometres, preferably 455 nanometres wavelength.

### REFERENCE NUMERAL LIST

1 air purifying device

2 housing

3 air inlet channel

4 air outlet channel

5, 7 air purifying unit

51, 71 non-woven material

52, 72 region permeated with activated carbon

53, 73 photocatalytically active surface region

6, 26 lighting unit

8 air guide elements

9 air flow

10 fan

11 mount(s)

12 air feed channel

13 air guide channel

1-16. (canceled)

17. An Air purifying device comprising:
a housing having at least one inlet opening for supply of an air flow and at least one outlet opening for conducting out the air flow supplied by way of the inlet opening;
at least one air purifying unit and at least one lighting unit arranged in the housing, wherein the at least one air purifying unit takes on substantially the form of the cross-section of the housing and is arranged in the housing to almost fill out the cross-sectional area, wherein the at least one lighting unit is arranged in the housing to be opposite the at least one air purifying unit, and the at least one air purifying unit is formed to be permeable to air and has at least one region which is permeated with activated carbon and which is surrounded at least partly by a non-woven material, wherein at least one photocatalytically active surface region in or at the non-woven material is present on the side of the at least one air purifying unit facing the lighting unit, wherein the air flow in the housing, at least in part, flows around or through the at least one photocatalytically active surface region and the surface region permeated by activated carbon, wherein the at least one photocatalytically active surface region of the at least one air purifying unit can be irradiated with light by the at least one lighting unit.

18. The air purifying device according to claim 17, wherein the at least one photocatalytically active surface region is coated at least partly with a metal oxide or a mixed oxide, or is packed or permeated with a metal oxide or a mixed oxide.

19. The air purifying device according to claim 18, wherein the metal oxide is an oxide of a transition metal.

20. The air purifying device according to claim 18, wherein the metal oxide or mixed oxide is of CuO, Co₃O₄, CoOₓ, NiO, MnOₓ, MnO₂, MnO₃, ZnO, Fe₂O₃, WO₃, CeO₂, TiO₂, Al₂O₃, V₂O₃, ZrO₂, HfO₂, Dy₂O₃, Cr₂O₃ and/or Nb₂O₅.

21. The air purifying device according to claim 17, wherein the at least one air purifying unit is fixable to at least one inner wall of the housing.

22. The air purifying device according to claim 17, wherein the at least one lighting unit is arranged in the housing of the at least one air purifying unit to be opposite the at least one photocatalytically active surface region of the at least one air purifying unit on one or more inner walls of the housing or approximately in the middle of the housing, wherein the inner walls of the housing are coated in the region between the at least one air purifying unit and the at least one lighting unit with a light-reflective material.

23. The air purifying device according to claim 17, wherein lenses which focus the light emitted by the at least one lighting unit onto the at least one air purifying unit are arranged in front of the at least one lighting unit, or light reflectors or light-reflective regions are arranged behind and/or adjacent to the at least one lighting unit and reflect the light, which is emitted by the at least one lighting unit, in the direction of the at least one air purifying unit.

24. The air purifying device according to claim 17, wherein the inner walls of the housing are painted white or coated with a light-reflective material.

25. The air purifying device according to claim 17, wherein air guide elements deflecting the air flow diffusely in the housing are arranged in the housing so that the air flow impinges almost uniformly on the entire surface of the non-woven material of the at least one air purifying unit and flows therethrough or the air flow is substantially directed onto the at least one photocatalytically active surface region and flows therearound or therethrough.

26. The air purifying device according to claim 17, wherein the at least one air purifying unit has a frame of plastics material or metal which is enclosed by the non-woven material at least at one side and a receiving region for granulate materials is present behind the non-woven material, wherein this receiving region is filled with activated carbon or a mixture of activated carbon and granulate coated with titanium dioxide (TiO₂) or packed with titanium dioxide ions (TiO₂ ions).

27. The air purifying device according to claim 17, wherein threads or fibres doped with titanium dioxide ions (TiO₂ ions) are worked or introduced into the non-woven material at least in part, which forms the at least one photocatalytically active surface region, or regions of the non-woven material are doped with titanium dioxide ions (TiO₂ ions).

28. The air purifying device according to claim 17, wherein the surface of the at least one air purifying unit with the non-woven material at least at the side having the at least one photocatalytically active surface region is formed to be wave-shaped or has conical, folded, cylindrical, frusto-conical, frusto-pyramidal, spherical or hemispherical geometric shaping.

29. The air purifying device according to claim 17, wherein the at least one lighting unit is controllable in the light intensity thereof, wherein an air quality measuring sensor arranged in the air inlet channel or at the air outlet channel determines the air quality and a control unit controls the light intensity of the at least one lighting unit on the basis of the air quality determined by the air quality measuring sensor.

30. The air purifying device according to claim 17, wherein the at least one lighting unit is so controllable in the light intensity thereof by the control unit that the control unit controls the light intensity of the at least one lighting unit in dependence on the rotational speed of a fan arranged upstream of the air inlet channel or the air outlet channel.

31. The air purifying device according to claim 17, wherein the air purifying device is arranged in the air intake of the air circulation flap of a motor vehicle.

32. The air purifying device according to claim 17, wherein the air purifying device comprises a volatile organic compound sensor for control of the air purifying device appropriately to requirements.
